(19) United States
(12) Patent Application Publication
(10) Pub. No.: US 2004/0050680 A1
(43) Pub. Date: Mar. 18, 2004

(54) Heterogeneously catalyzed reactive distillation in the suspension mode

(76) Inventors: Christian Miller, Ruppertsberg (DE); Gerd Kaibel, Lampertheim (DE); Hardo Siegel, Speyer (DE)

Correspondence Address: Herbert B. Keil, KEIL & WEINKAUF, 1350 Connecticut Ave., N.W., Washington, DC, 20036, US

(21) Appl. No.: 10/619,439

(22) Filed: Jul. 16, 2003

(30) Foreign Application Priority Data
Aug. 3, 2002 (DE) 10235552.5

(51) Int. Cl. C07C 43/00; B01D 3/00

(52) U.S. Cl. 203/99; 568/697

(57) ABSTRACT

Process for carrying out a reactive distillation in the presence of a heterogeneous catalyst which is suspended as disperse phase in the liquid, wherein part of the catalyst-containing suspension is continuously discharged from the column and is treated in a work-up stage and at least part of the work-up catalyst is returned to the column.

The present invention relates to an improved process for carrying out a reactive distillation in the presence of a heterogeneous catalyst which is suspended as disperse phase in the liquid.

Various methods have become established in industry for carrying out heterogeneously catalyzed reactive distillations.

One possibility is to apply the active catalyst composition directly to the packing. Forms of packing used here correspond to the construction types generally customary in distillation technology. An example which may be mentioned is KATAPAK-M® from Sulzer AG. However, a problem which occurs in industrial use is that many catalytically active compositions cannot be applied to such packing in such a way that the required abrasion resistance is obtained. This leads to a deterioration in the economics of these processes and at the same time increases the process engineering complexity required.

For this reason, packing in which conventional catalysts in the form of larger bodies are used is employed more widely. The catalysts are, for example, accommodated in wire mesh pockets. These pockets can either serve directly as distillation internals, as is the case for, for example, KATAPAK-M® from Sulzer AG, or the flat pockets are installed between the individual layers of the distilllation packing (e.g. mesh packing for separation of substances), as is the case for, for example, Multipak® packing from Montz GmbH. However, the use of such packing is susceptible to malfunctions, since the matching liquid trickle densities have to be adhered to precisely, which often proves to be difficult in practice.

The use of “bales” from CDTech, Houston, USA, is described in, for example, EP-A 466954. Here too, the catalyst is fixed in a type of pocket structure within the column. Compared to the pockets described above, the pocket structure is coarser, which leads to a reduction in the number of theoretical plates which can be achieved per meter of column height.

A general problem in all systems in which the catalyst is sewn into pockets is that removal of the exhausted catalyst and introduction of a fresh charge is very cumbersome and time-consuming.

When using tray columns, various possible ways of accommodating the catalyst in downcomers or on the tray have been described. Suspended catalysts in tray columns represent a particular form. Here, the catalyst is present in suspension on the individual trays and is held back on each tray by means of filter elements, which is complicated in terms of construction and process engineereing. Such internals are described, for example, in DE 19808385 and in U.S. Pat. No. 5,308,451. In the process described in U.S. Pat. No. 4,471,145, the catalyst is suspended on the respective trays and is held back there.

In the process indicated, removal of the exhausted catalyst and introduction of a fresh charge are time-consuming and complicated in process engineering terms. Furthermore, it is not possible to remove and replace the catalyst during operation.

It is an object of the present invention to find a process which avoids the disadvantages mentioned. Furthermore, it should make it possible to use conventional internals, having a simple construction.

We have found that this object is achieved by a process for carrying out a reactive distillation in the presence of a heterogeneous catalyst which is suspended as disperse phase in the liquid, in which part of the catalyst-containing suspension is continuously discharged from the column and is treated in a work-up stage and at least part of the worked-up catalyst is returned to the column.

In an alternative process, the catalyst taken off is not returned and fresh catalyst is added only when required.

The process of the present invention offers the advantage that the catalyst can be worked up and, if appropriate, returned as required in a simple manner during operation, which improves the economics the process.

It is particularly advisable to use the catalyst in the form of a finely divided suspended catalyst as disperse phase in the liquid. As catalysts, it is possible to use all catalysts known from the prior art which are suitable for a suspension process. Generally suitable types of catalyst are, for example, metal, precipitated, supported or Raney-type catalysts whose preparation is described, for example, in Ullmann, Enzyklopädie der Technischen Chemie, 4th edition, 1977, Volume 13, pages 558 to 665. In the case of unsupported catalysts, preference is given to using metal catalysts, particularly preferably noble metal catalysts, for example platinum, rhodium, palladium, cobalt, nickel or ruthenium. Apart from the metals of transition group VII of the Periodic Table, it is also possible to use the metals of main groups I and VII, preferably copper and/or rhenium. Furthermore, metal salts and oxides, e.g. rhenium sulfides, copper sulfides, zinc chromites, copper chromites, nickel oxides, molybdenum oxides, aluminum oxides, rhenium oxides and zinc oxides, can also be used.

Raney-type catalysts such as Raney nickel, Raney copper, Raney cobalt, Raney nickel/molybdenum, Raney nickel/copper, Raney nickel/chromium, Raney nickel/chromium/iron or rhenium sponge can also be used very advantageously in the process of the present invention. The preparation of a Raney nickel/molybdenum catalyst is described, for example, in U.S. Pat. No. 4,153,578.

In the process of the present invention, particular preference is given to using supported suspension catalysts. As support materials, it is possible to use all support materials known in catalyst production, particularly preferably activated carbon, silicon carbide, aluminum oxide, silicon oxide, silicon dioxide, titanium dioxide, zirconium oxide, magnesium oxide, zinc oxide, calcium carbonate, barium sulfate or mixtures thereof. As active components in the support suspension catalysts, it is in principle possible to use all metals, preferably metals of transition group VIII of the Periodic Table, e.g. platinum, rhodium, palladium, cobalt, nickel, ruthenium or mixtures thereof. It can likewise be advantageous to use the metals of main groups I, III and VII of the Periodic Table, preferably copper and/or rhenium, and also yttrium and the elements of the lanthanide series, preferably lanthanum and/or praseodymium.

The active component is generally present in the supported suspension catalysts in an amount of from 0.001 to 30% by weight, preferably from 0.01 to 8% by weight, based on the total weight of the catalyst.

The particle size of the catalyst is usually in a range from about 0.1 to 500 μm, preferably from about 0.5 to 200 μm, particularly preferably from about 1 to 100 μm. The particle size is reduced over time as a result of mechanical stress caused by pumping of the suspension until a limiting particle size of about 1 μm is reached.

In a preferred embodiment of the process of the present invention, packing is installed in the column and finely divided suspension catalysts are allowed to flow over these separation internals together with the liquid. Since the catalyst is no longer assigned to a particular theoretical plate, but instead flows over the entire desired length of the column, it only needs to be taken off at one point, for example at the bottom of the column or in the middle region of the column, and separated off by, for example, filtration and if appropriate returned again later. Substreams can be discharged and, if required, passed to regeneration. The catalyst can be separated off either within or outside the column.

It can be particularly advisable to use internals such as structured packings, irregular beds, a knitted mesh fabric or an open-celled foam structure, preferably made of plastic (e.g. polyurethane or melamine resin), or ceramic in the region of the column in which the catalyst is located, so that the suspension flows over these during operation. Preference is given to using structured packing, for example packing made of wire mesh, sheet metal or expanded metal, in the process of the present invention. In a preferred embodiment, packing material and/or geometry of the packing are chosen so that partial reversible adhesion of the catalyst particles to the packing is achieved. The choice is in each case dependent on the substances used and on the boundary conditions and can be determined by a person skilled in the art by means of routine tests. In general, the procedure is similar to the determination of the dynamic holdup in a distillation column. Here, the outlet and inlet of the column are simultaneously closed during steady-state continuous operation. The amount of catalyst in the suspension in the column and on the internals is subsequently determined. The amount of catalyst per reaction volume determined in this way should be greater than the total amount of catalyst divided by the total amount of liquid present in the liquid circuit. The partial adhesion advantageously limits the amount of catalyst which has to be circulated and an acceleration of the reaction is achieved as a result of the relative motion of catalyst particles and working solution. Examples of packing suitable for this purpose are wire mesh packing having a high specific surface area, as is supplied by Montz under the designation A3 or Sulzer under the designations DX, BX or EX, which have been additionally roughened. Likewise suitable is additionally roughened sheet metal packing, with or without perforations. When perforations are used, they should be kept appropriately small. Examples of sheet metal packing are the types Montz B1 and BSH and Sulzer Mellapack. Overall, the internals should have surface roughnesses in the range from 0.1 to 10 times, preferably from 0.5 to 5 times, the mean particle size of the suspended catalyst particles.

Apart from the use of structured packing, it can also be useful to employ trays having wide openings in the process of the present invention. For the present purposes, “wide openings” are openings of from 0.5 to 50 nm, preferably from 1 to 20 nm. The gaseous, liquid and solid components of the working solution flow through the relatively wide openings without blocking of the openings occurring. The narrowing of the cross section when the suspension flows through the openings advantageously results in relative motion of the catalyst particles and the working solution and thus in an acceleration of the reaction. For example, dual-flow trays or sieve trays as are also employed in liquid-liquid extractors are suitable.

A substream of the catalyst-containing suspension is preferably taken off in the middle region of the column or at or close to the bottom of the column. Taking the substream off in the middle region of the column is advantageous whenever high residence times and high temperatures in the liquid phase lead to secondary reactions or whenever the desired product is to be freed of or depleted in high-boiling components. The catalyst can be separated from the liquid by separation methods such as filtration, flotation or sedimentation. Crossflow filtration is particularly useful. If required, the catalyst can be passed to a generally known regeneration and subsequently be returned to the column. The catalyst is preferably returned in the middle region of the column or in the upper part of the column. Discharge and return of the catalyst can advantageously be carried out during operation of the column. The substream returned to the column is preferably small, so that the internal flows in the column with recirculated catalyst are not more than five times, preferably not more than two times, the internal flows without return of the catalyst to the column. Furthermore, fresh catalyst can also be introduced into the column by means of the circulating stream.

When using a column which is equipped with dividing devices which are effective in the longitudinal direction, e.g. push-in metal sheets or a welded-in wall, a plurality of reaction products having different boiling points can simultaneously be obtained in pure form.

It can also be advisable for the catalyst separated off in the circuit, e.g. as filter cake having a very low residual moisture content, to be redispersed in order to minimize backmixing with starting materials.

The process of the present invention is particularly suitable, for example, for esterifications, acetal formations, etherifications, aldolizations and hydrogenations. Advantages are obtained particularly when large catalyst areas are required to increase the space-time yields, because these are not present in the case of coarse catalyst particles.

An example of such a reaction is the aldolization of citral and acetone to give pseudoionone.

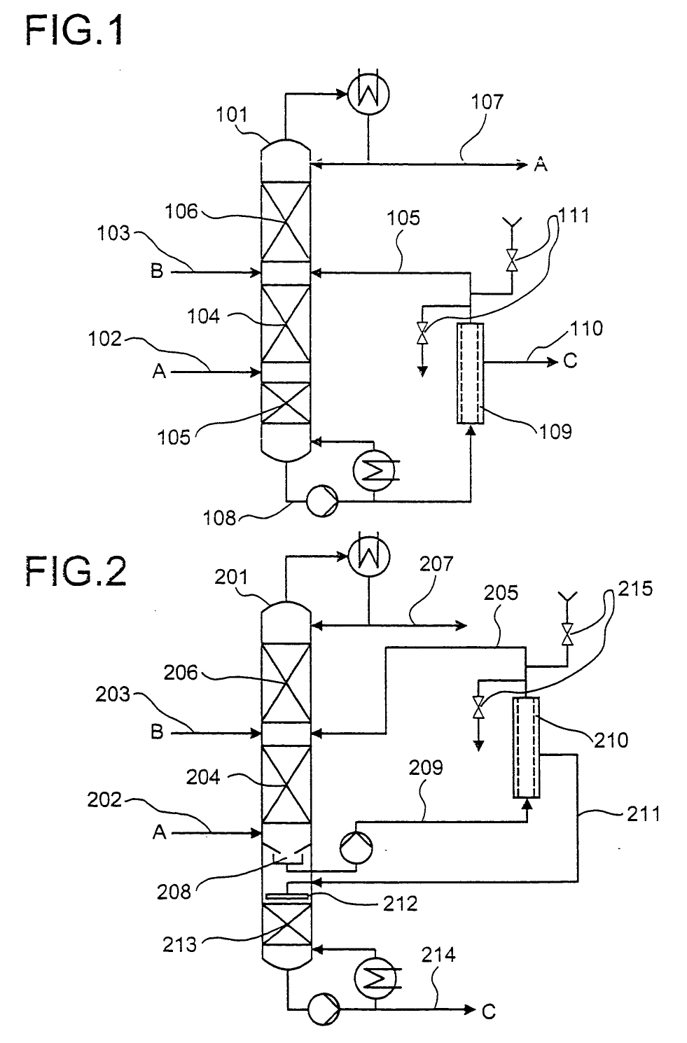

The process of the present invention is described in more detail by way of example with the aid of FIGS. 1 to 3.

FIG. 1 shows a fractionation column (101) functioning as reaction column. The starting materials A and B are fed in via the feed streams (102) (starting material A) and (103) (starting material B) and react further to form the product C. The boiling points of the substances A, B and C increase in that order. Internals (104) are installed in the reaction column. It is advantageous to feed the higher-boiling reactant separately and continuously into the reaction column (hereinafter referred to simply as the “column”) at a point above that at which the lower-boiling reactant is introduced, since countercurrent flow of the reactants is effected in this way.

The lower-boiling reactant is preferably fed into the column in gaseous or superheated form if this avoids decomposition in the liquid phase in the column at high temperatures and the formation of by-products.

The catalyst suspended in the liquid is likewise fed into the column at a point above the internals (104) by means of line (105) and flows over the internals. Preference is given to using roughened wire mesh which achieves partial reversible adhesion of the particles to the packing. This makes it possible to obtain an increased concentration of catalyst in the transport zone defined by the internals. In this zone, the starting materials come into contact with the catalyst, so that they react to form the reaction product C. The adhesion of the catalyst to the internals at the same time advantageousely results in a high relative velocity of the catalyst and the liquid, which favors mass transfer.

Above the catalyst transport zone defined by the internals, there is a distillation zone (106) in which distillation separation elements have been installed. This distillation zone ensures that the starting material B added via the upper feed stream (103) does not get into the distillate. The starting material A which is introduced in excess is separated off as distillate via line (107) and can be returned to the column in the lower region via line (102).

Below the internals (104) there is the zone (105) in which the catalyst suspended in the liquid is transported to the bottom of the column. At the same time, this zone can fulfill distillation functions by depleting the reaction product C in low-boiling components, in particular the starting material A introduced via line (102).

A substream of the stream (108) taken off at the bottom of the column is branched off and the suspended catalyst is preferably separated from the reaction product C by crossflow filtration (109). Here, the reaction product is obtained as permeate (110). The catalyst suspended in a substream of the reaction medium is recirculated via line (105) and fed back into the column above the internals (104). Fresh catalyst can be fed in or exhausted catalyst can be taken off as required during operation by means of line (111).

FIG. 2 shows a fractionating column (201) functioning as reaction column. The starting materials A and B are fed in via the feed streams (202) (starting material A) and (203) (starting material B) and react further to form the product C. The boiling points of the substances A, B and C increase in that order. Internals (204) are installed in the reaction column. The catalyst suspended in the liquid is likewise fed into the column at a point above the internals (204) by means of line (205) and flows over the internals. The internals produce a defined transport zone, as a result of which an increased concentration of catalyst can be achieved. In this zone, the starting materials come into contact with the catalyst, so that they react to form the reaction product C. The adhesion of the catalyst to the internals at the same time advantageously results in a high relative velocity of the catalyst and the liquid, which favors mass transfer.

Above the catalyst transport zone defined by the internals, there is a distillation zone (206) in which distillation separation elements have been installed. This distillation zone ensures that the starting material B introduced via the upper feed stream (203) does not get into the distillate. The starting material A which has been introduced in excess is separated off as distillate via line (207) and can be fed back into the column in the lower region via line (202). In the liquid collector (208), which is located directly below the internals (204), the liquid is collected and is passed via line (209) to the filtration (210). The permeate (211) is returned to the column via a distributor (212) above the zone (213). This zone (213) fulfills distillation functions by depleting the reaction product C in relatively low-boiling components, in particular the starting material A fed in via line (202). The reaction product C is obtained as bottom stream (214) from the column.

The suspended catalyst is preferably separated from the reaction product C by crossflow filtration (210). The catalyst suspended in a substream of the reaction medium is recirculated via line (205) and returned to the column above the internals (204). Depending on requirements, fresh catalyst can be fed in or exhausted catalyst taken out by means of line (215) during operation of the column.

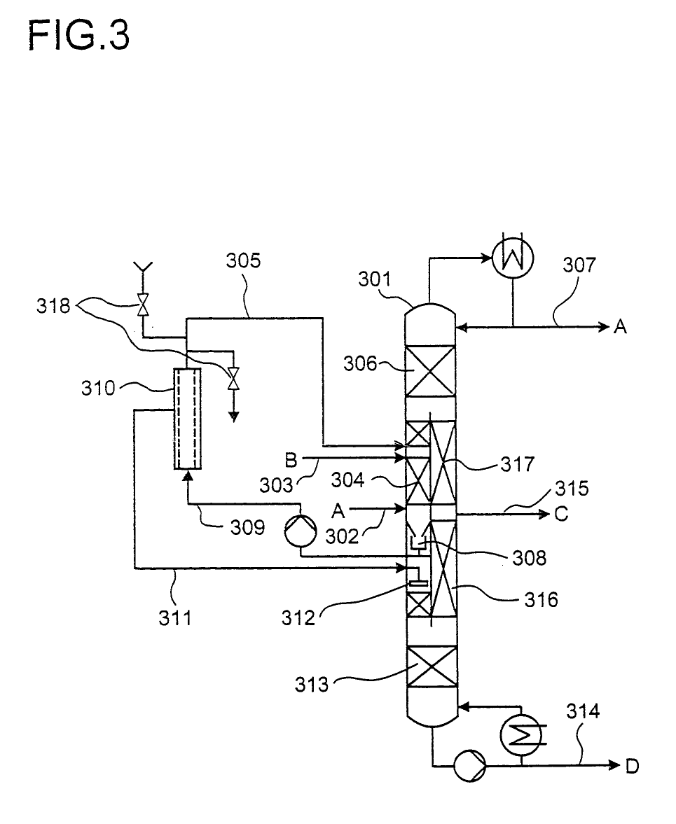

FIG. 3 shows a fractionating column (301) functioning as reaction column. The starting materials A and B are fed in via the feed streams (302) (starting material A) and (303) (starting material B) and react further to form the products C and D. The boiling points of the substances A, B, C and D increase in that order. The column is divided by a dividing device which is effective in the longitudinal direction and extends above and below the feed points for the starting materials A and B. The catalyst suspended in the liquid is, like the starting material B, fed into the column at a point above the internals (304) by means of line (305) and flows over the internals. The internals produce a defined transport zone, as a result of which an increased concentration of catalyst can be achieved. In this zone, the starting materials come into contact with the catalyst, so that they react to form the reaction products C and D. The adhesion of the catalyst to the internals at the same time advantageousely results in an increased relative velocity of the catalyst and the liquid, which favors mass transfer.

Above the catalyst transport zone defined by the internals, there is a distillation zone (306) in which distillation separation elements have been installed. This distillation zone ensures that the reaction product C does not get into the distillate. The starting material A which has been introduced in excess is separated off as distillate via line (307) and can be fed back into the column in the lower region via line (302). In the liquid collector (308), which is located directly below the internals (304), the liquid is collected and is passed via line (309) to the filtration (310). The permeate (311) is returned to the column via a distributor (312) above the zone (313). The reaction product D is, in accordance with the order of boiling points, obtained as bottom stream (314). This zone (313) fulfills distillation functions by depleting the reaction product D in relatively low-boiling components, in particular the low-boiling reaction product C. The reaction product C itself can be taken off in very pure form via line (315). This is achieved by means of the dividing device which is effective in the longitudinal direction and the distillation zones (316) and (317). Owing to its boiling point which is between that of the starting material A and that of the reaction product D, the reaction product goes into both the distillation zone (316) and the distillation zone (317). While the reaction product C is being depleted in low-boiling components, in particular the reaction product D, in the distillation zone (316), low-boiling components, in particular the starting material A, are separated off from the product stream from the reaction in the distillation zone (317). The dividing device which is effective in the longitudinal direction prevents transverse mixing of the liquid and vapor streams from the zone (304) with the liquid and vapor streams from the zones (316) and (317).

The suspended catalyst is preferably separated from the reaction product C by crossflow filtration (310). The catalyst suspended in a substream of the reaction medium is recirculated via line (305) and returned to the column above the internals (304). The catalyst is advantageously transported through the column only in the region of the internals (304). Depending on requirements, fresh catalyst can be fed in or exhausted catalyst taken out by means of line (318) during operation of the column.

### EXAMPLE

Aldol Condensation of Acetone and Citral to Give Pseudoionone

The reaction of acetone with citral was carried out in an experimental column which corresponded to that shown schematically in FIG. 2. The column had a diameter of 0.055 m and was provided in the upper region (206) and in the lower region (213) with wire mesh packing of the type A3-500 from Montz GmbH, Hilden, over a height of 0.6 m in each case. The region (204), on the other hand, was provided over a height of 0.6 m with roughened wire mesh packing of the type A3-1200 from Montz GmbH, Hilden. 55 g/h of citral were fed in continuously as feed stream (203) and 210 g/h of acetone were fed continuously into the column as feed stream (202). At the top of the column 196 g/h of distillate consisting of 96.2% of acetone, 0.4% of diacetone alcohol, 0.2% of mesityl oxide and 3.2% of water were taken off.

The catalyst suspension was fed in together with the recirculated product stream via the feed line (205). As suspension catalyst, use was made of a praseodymium-coated aluminum oxide catalyst in powder form. The praseodymium content was 5% by weight. The suspension introduced via the feed line (205) had a solids content of 20% by mass.

The liquid was collected in the liquid collector (208) located directly above the internals (204) and was passed via line (209) to the crossflow filtration (210). The liquid comprised about 64.2% by weight of acetone, 0.2% by weight of water, 0.1% by weight of mesityl oxide, 0.3% of diacetone alcohol, 31.5% by weight of pseudoionone, 1.3% of citral and 0.5% of high boilers. The solids content was about 5% by mass. The filtration was carried out using a filtration unit similar to the commercially available filter module from Membraflow, Aalen-Essingen.

The permeate (211) was returned via a distributor (212) above the zone (213). 69 g/h of crude product consisting of about 94.4% of pseudoionone, 4.0% of citral and 1.6% of high boilers was taken from the column as bottom stream.

We claim:

1. A process for carrying out a reactive distillation in the presence of a heterogeneous catalyst which is suspended as disperse phase in the liquid, wherein
part of the catalyst-containing suspension is continuously discharged from the column and
is treated in a work-up stage and
at least part of the worked-up catalyst is returned to the column.

2. A process as claimed in claim 1, wherein the catalyst is discharged from the column during operation of the column.

3. A process as claimed in claim 1 or 2, wherein the worked-up and/or fresh catalyst is fed into the column during operation of the column.

4. A process as claimed in any of claims 1 to 3, wherein structured packing is used as internals in the region in which the catalyst is located in the column.

5. A process as claimed in claim 4, wherein some of the catalyst particles adhere reversibly to the packing used.

6. A process as claimed in claim 5, wherein wire mesh is used as packing.

7. A process as claimed in claim 6, wherein roughened wire mesh is used.

8. A process as claimed in any of claims 1 to 3, wherein trays having wide openings are used in the region in which the catalyst is located in the column.

9. A process as claimed in any of claims 1 to 8, wherein the catalyst is separated off in the middle region of the column and is later fed back in.

10. A process as claimed in any of claims 1 to 8, wherein the catalyst is separated off at the bottom of the column and is later fed back in.

11. A process as claimed in any of claims 1 to 10, wherein separation devices which are effective in the longitudinal direction are installed in the column.

12. A process as claimed in any of claims 1 to 11, wherein the catalyst is separated off by filtration, flotation or sedimentation.

13. A process as claimed in claim 12, wherein a crossflow filtration is carried out as filtration.

14. A process as claimed in any of claims 1 to 12, wherein the catalyst which has been separated off is regenerated by washing with a caustic alkali, a base or a solvent, by calcination or by another method.

15. The use of a process as claimed in any of claims 1 to 14 for esterifications, acetal formations, etherifications, aldolizations or hydrogenations.

16. The use of a process as claimed in any of claims 1 to 14 for the aldolization of acetone and citral to form pseudoionone.
